(19) United States
(12) Patent Application Publication
(10) Pub. No.: US 2024/0207369 A1
(43) Pub. Date: Jun. 27, 2024

(54) In Vitro Drugs from Prodrugs and Use Thereof

(71) Applicant: Brandenburgische Tehnische Universitat Cottbus-Senftenberg, Cottbus (DE)

(72) Inventors: Friedrich Jung, Dresden (DE); Jan-Heiner Kupper, Kleinkoschen (DE); Katrin Scheibner, Senftenberg (DE)

(21) Appl. No.: 18/288,094

(22) PCT Filed: Apr. 25, 2022
(86) PCT No.: PCT/EP2022/060814

§ 371 Date: Oct. 24, 2023

(30) Foreign Application Priority Data
Apr. 25, 2021 (DE) 10 2021 204 094.2

(51) Int. Cl. A61K 38/44; A61K 31/138; A61K 31/4365

(52) U.S. Cl. CPC A61K 38/44; A61K 31/138; A61K 31/4365

(57) ABSTRACT

An object of the invention is a process for the preparation of a pharmaceutical, wherein a prodrug with bioactivation by cytochrome P450 enzymes is mixed with at least one peroxygenase, and the product obtained is isolated. Furthermore, the invention relates thereto to selected prodrugs, such as thienopyridines and the products obtained according to the invention and their use.

An object of the invention is a process for the preparation of a pharmaceutical, wherein a prodrug, i.e. such a prodrug with bioactivation by cytochrome P450 enzymes, is mixed with at least one peroxygenase and the product obtained is isolated.

Furthermore, the invention relates thereto to selected prodrugs, in particular to such prodrugs as thienopyridines, and to the products obtained according to the invention and their use.

There is a high need for biotechnological production of metabolites, but in vitro and not in vivo from a prodrug, which are mainly produced in liver cells (hepatocytes) or in the liver during biotransformation or metabolization.

The liver is a central detoxification organ of the metabolism. Liver cells (hepatocytes) represent 70-80% of all cells in the liver and have the important physiological liver functions.

The liver uses biotransformation or metabolization to excrete or detoxify ingested substances (e.g. drugs, toxins, natural products). The phase I enzymes of the cytochrome P-450 (CYP450) system are particularly important for biotransformation. The CYP450 enzymes are oxidoreductases that cause oxidative degradation or metabolization of numerous substances, including drugs. Of the numerous CYP450 isozymes with varying substrate specificity that exist in humans, the CYP1A2, -2C9, -2C19, -2D6, -2E1, and -3A4 isozymes alone are responsible for approximately 90% of all oxidative metabolism of drugs. In many cases, it is only through these biochemical changes that a large number of drugs acquire their curative efficacy or even cause toxic metabolites with adverse drug effects.

Such curative liver metabolites can be produced in sufficient quantities, and in particular these liver metabolites mostly exhibit high regioselective and stereoselective specificity as stereoisomers. Such regioselective as well as stereoselective modifications are characteristic of biotransformative liver enzymes.

Phase I enzymes have been described for biotransformation or metabolization in liver cells, in particular the cytochrome P-450 (CYP450) system, so-called oxidoreductases.

It is further noteworthy for the present invention that such biotransforming enzymes may also be present in other organisms, such as fungi and bacteria, for evolutionary reasons.

WO 2008/119780 A2 describes a process for the enzymatic hydroxylation of non-activated hydrocarbons, in particular aromatic rings of non-activated hydrocarbon molecules (for example, the selective conversion of naphthalene to 1-naphthol) by using fungal peroxidases from basidiomycetes of the family Bolbitiaceae (e.g., Agrocybe sp.) for the production of pharmaceuticals, terpenes, steroids or fatty acids.

DE 102008034829 A1 discloses a one-step enzymatic process for the regioselective hydroxylation of 2-phenoxypropionic acid to 2-(4-hydroxyphenoxy)propionic acid. The enantioselective and regioselective monohydroxylation of 2-phenoxypropionic acid to 2-(4-hydroxyphenoxy)propionic acid by isolated biocatalysts (in vitro) can also be highly regioselectively converted by a stable extracellular fungal enzyme, Agrocybe aegerita peroxygenase (AaP), 2-phenoxypropionic acid to 2-(4-hydroxyphenoxy)propionic acid and preferably to its (R)-enantiomer.

WO 2015071264 A1 describes the production of biogenic substances, but using human liver cells.

However, the prior art does not describe the biotechnological production of liver-specific metabolites from a prodrug and its use as a drug.

In the prior art, the disadvantage is that especially liver-specific human metabolites cannot be produced in sufficient yield and diversity. Moreover, in the prior art, precursor molecules are mostly synthesized. Often, additional semi-synthetic processes are required, so that the necessary further regio- and stereoselective modifications have to be laboriously introduced into the substance(s), which are crucial for the pharmacological efficacy of the metabolites from the liver.

The invention sets out to provide suitable metabolites from a prodrug in vitro and in this way to prepare and provide a suitable drug.

Particularly advantageously, these drugs can be provided to patients whose P450 enzyme complement is impaired by mutations or polymorphism. For example, approximately 20-40% of patients respond less or not at all to therapy with the liver-specific prodrug clopidogrel (a thienopyridine) due to mutations in the CYP2C19 gene or CYP2C19 genotypes (Brandt et al, Common polymorphisms of CYP2C19 and CYP2C9 affect the pharmacokinetic and pharmacodynamic response to clopidogrel but not prasugrel J. Thromb. Haemost., 2007, 5(12), 2429-2436).

The invention therefore relates to the biotechnological production of metabolites from a prodrug, which are produced according to a new process.

The posed task is solved by at least one patent claim from the conveyed technical teaching.

It is therefore an object of the invention to provide a process for the preparation of a pharmaceutical composition, wherein a prodrug having bioactivation by cytochrome P450 enzymes is mixed with at least one peroxygenase and the product obtained is isolated.

Surprisingly, metabolites, in particular liver-specific human metabolites, can be continuously produced in high yields at low cost, and, moreover, stereoselective compounds of such metabolites can advantageously be obtained, although, for example, non-human peroxygenases are used. Therefore, the invention relates to a new method for regioselective, enzymatic oxyfunctionalization of a liver-specific prodrug or a prodrug with bioactivation by cytochrome P450 enzymes.

The process according to the invention causes regioselective oxyfunctionalization of the prodrug in the presence of an oxidizing agent, preferably in an aqueous medium.

In a preferred embodiment of the invention, the prodrug with bioactivation by cytochrome P450 enzymes is present free as an isolated substance or pure substance and in any case not in a liver cell.

The term “prodrug with bioactivation by cytochrome P450 enzymes” means that a prodrug is present, wherein the prodrug is mostly activated in the liver by cytochrome P450 enzymes, in particular those such as CYP1A1, CYP1A2, CYP2A6, CYP2B6, CYP2C8, CYP2C9, CYP2C19, CYP2D6, CYP2E1, CYP3A4, CYP3A5, CYP3A7, CYP4A11, CYP1B1, CYP2A13, CYP2F1, CYP2J2, CYP2R1, CYP2S1, CYP2U1, CYP2W1, CYP3A43, and metabolization to metabolites occurs mostly in the liver, particularly in the form of a drug. Cytochrome P450 enzymes are heme proteins with enzymatic activity (oxidoreductases) and mostly act as monooxygenases. Preferred prodrugs according to the invention are those that metabolize in the liver to a drug.

These metabolites obtained represent a “drug” from the “prodrug” and relate to a drug according to the invention. “Prodrugs with bioactivation by cytochrome P450 enzymes” are described in the literature or can be easily determined using an assay in vitro (M. Huttunen and Jarkko Rautio, Prodrugs—An Efficient Way to Breach Delivery and Targeting Barriers Kristiina Current Topics in Medicinal Chemistry, 2011, 11, 2265-2287).

According to the invention, therefore, particularly such prodrugs with bioactivation by cytochrome P450 enzymes are included, such as:

### A

Amiodarone (metabolized by CYP3A4 and CYP2C8 to desethylamiodarone), Latini R, Tognoni G, Kates R E: Clinical pharmacokinetics of amiodarone. Clin Pharmacokinet. 1984 March-April;9(2):136-56. doi: 10.2165/00003088-198409020-00002. [PubMed:6370540],
Aripiprazole lauroxil (activation by CYP3A4 and CYP2D6: https://go.drugbank.com/drugs/DBO1238,

### B

Bambuterol (activated by CYP2D6): Br J Clin Pharmacol. 1998 May; 45(5): 479-484. doi: 10.1046/j.1365-2125.1998.00697.x,
Brincidofovir (CYP metabolization shown): Viruses 2010, 2, 2740-2762; doi:10.3390/v2122740,

### C

Carbamazepine (activated by CYP3A4): https://go.drugbank.com/drugs/DB00564,
Carisoprodol (activated by CYP2C19): https://go.drugbank.com/drugs/DB00395,
Clomifene (activated by CYP enzymes): YAKUGAKU ZASSHI130(10) 1325-1337 (2010) The Pharmaceutical Society of Japan,
Clopidogrel (activated by CYP2C19, CYP1A2, and CYP2B6): https://go.drugbank.com/drugs/DB00758,
Codeine (activated by CYP2D6 and CYP3A4): https://go.drugbank.com/drugs/DB00318,
Cyclophosphamide (activated by CYP3A4, CYP2B6): Preissner et al, Advances in Pharmacology, Volume 74 Copyright #2015 Elsevier Inc. ISSN 1054-3589, http://dx.doi.org/10.1016/bs.apha.2015.03.004,

### D

Decarbazine (activated by CYP1A2): Preissner et al, Advances in Pharmacology, Volume 74 Copyright #2015 Elsevier Inc. ISSN 1054-3589, http://dx.doi.org/10.1016/bs.apha.2015.03.004,
N-desmethyltamoxifen (activated by CYP2D6 to form endoxifen): https://go.drugbank.com/drugs/DB0067,
Desogestrel (activated by CYP2C9 and CYP2C19): Br J Clin Pharmacol 60:169-75, 2005; DOI:10.1111/j.1365-2125.2005.02382.x,
Dextromethorphan (activated by CYP2D6): https://pubchem.ncbi.nlm.nih.gov/compound/5360696 #section=Drug-Warnings,
N,N-didesmethyltamoxifen (activated by CYP2D6 and forms endoxifen): https://go.drugbank.com/drugs/DB00675,

### E

Ellipticine (activated by CYP1A1/2 and 3A4): Int J Mol Sci. 2014 Dec. 25; 16(1):284-306. doi: 10.3390/ijms16010284,
Enalapril (activated by CYP3A4): https://proteopedia.org/wiki/index.php/Enalapril,

### F

Fludrocortisone (activated by CYP3A family): https://go.drugbank.com/drugs/DB00687,
Flutamide (activated by CYP1A2): https://go.drugbank.com/drugs/DB00499, Future Med Chem. 2013 February; 5(2): 213-228. doi:10.4155/fmc.12.197,

### I

ICT2700 (activated by CYP1A1 to duocarmycin), Future Med Chem. 2013 February; 5(2): 213-228. doi:10.4155/fmc.12.197,
Ifosfamide (activated by CYP3A4, -2B6): Preissner et al, Advances in Pharmacology, Volume 74 Copyright #2015 Elsevier Inc. ISSN 1054-3589, http://dx.doi.org/10.1016/bs.apha.2015.03.004,
Irinotecan (activated by CYP3A4, CYP3A5): Preissner et al, Advances in Pharmacology, Volume 74 Copyright #2015 Elsevier Inc. ISSN 1054-3589, http://dx.doi.org/10.1016/bs.apha.2015.03.004,

### L

Lansoprazole (activated by CYP3A4 to lansoprazole sulfone): https://go.drugbank.com/drugs/DB00448,
Leflunomide (activate by CYP1A2): https://go.drugbank.com/drugs/DB01097,
Levomethorphan (activated by CYP2D6): Molecules. 2018 September; 23(9): 2119. doi: 10.3390/molecules23092119,
Loratadine (activated by CYP3A4 and CYP2D6): Future Med Chem. 2013 February; 5(2): 213-228. doi:10.4155/fmc.12.197,
Losartan (activated by CYP2C9 and CYP3A4):Future Med Chem. 2013 February; 5(2): 213-228. doi:10.4155/fmc.12.197,
Lynestrenol (Metabolized to norethisterone by CYP2C9, CYP2C19, and CYP3A4): Korhonen T, Turpeinen M, Tolonen A, Laine K, Pelkonen O (2008). “Identification of the human cytochrome P450 enzymes involved in the in vitro biotransformation of lynestrenol and norethindrone”. J Future Med Chem. 2013 February; 5(2): 213-228. doi:10.4155/fmc.12.197, Steroid Biochem. Mol. Biol. 110 (1-2): 56-66. doi:10.1016/j.jsbmb.2007.09.025. PMID 18356043. S2CID 10809537,

### N

Nabumetone (activated by CYP1A2): Future Med Chem. 2013 February; 5(2): 213-228. doi:10.4155/fmc.12.197,

### P

Pantoprazoles (activated by CYP3A4 to desmethylpantoprazoles): https://go.drugbank.com/drugs/DB00213,
Parecoxib (metabolized by CYP3A4 & CYP2C9 to valdecoxib): https://go.drugbank.com/drugs/DB08439,
Prasugrel (activated by CYP3A4, CYP2C19, CYP2B6 and CYP2C9 to R-138727): Future Med Chem. 2013 February; 5(2): 213-228. doi:10.4155/fmc.12.197, Schror K, Siller-Matula J M, Huber K (2012) Pharmacokinetic basis of the antiplatelet action of prasugrel. Fundam Clin Pharmacol 26: 39-46,
Primidone (activated by CYP2C19 to phenobarbital): https://go.drugbank.com/drugs/DB00794,
Proguanil (activated by CYP2C19): https://www.pharmazeutische-zeitung.de/ausgabe-282012/potente-metaboliten/,

### S

Sibutramine (activated by CYP3A4 to desmethylsibutramine (M1; BTS-54354) and didesmethylsibutramine (M2; BTS-54505): https://go.drugbank.com/drugs/DB01105,

### T

Tafenoquine (activated by CYP2D6): Molecules 2020, 25, 884; doi:10.3390/molecules25040884,
Tamoxifen (Activation to endoxifen via CYP2D6, CYP3A4, and CYP3A5): Desta Z, Ward B A, Soukhova N V, Flockhart D A. Comprehensive evaluation of tamoxifensewuential biotransformation by the human cytochrome P450 system in vitro: prominent roles forCYP3A4 and CYP2D6. J. Pharmacol. Exp. Therap. 2004; 310:1062-1075. [PubMed: 15159443], Future Med Chem. 2013 February; 5(2): 213-228. doi:10.4155/fmc.12.197,
Tegafur (activated by CYP1A2, CYP2A6, and CYP2C8): Preissner et al, Advances in Pharmacology, Volume 74 Copyright #2015 Elsevier Inc. ISSN 1054-3589. http://dx.doi.org/10.1016/bs.apha.2015.03.004, Future Med Chem. 2013 February; 5(2): 213-228. doi:10.4155/fmc.12.197,
Temozolomide (activated by CYP3A4): Preissner et al, Advances in Pharmacology, Volume 74 Copyright #2015 Elsevier Inc. ISSN 1054-3589, http://dx.doi.org/10.1016/bs.apha.2015.03.004,
Ticlopidine (activated by CYP450 enzymes): Future Med Chem. 2013 February; 5(2): 213-228. doi:10.4155/fmc.12.197,
Tramadol (activated by CYP2D6): https://www.pharmazeutische-zeitung.de/ausgabe-282012/potente-metaboliten/,
Treosulfan (activated by CYP1A2 & CYP3A4): Preissner et al, Advances in Pharmacology, Volume 74 Copyright #2015 Elsevier Inc. ISSN 1054-3589, http://dx.doi.org/10.1016/bs.apha.2015.03.004,
Trofosamide (activated by CYP3A4 & CYP2B6): Preissner et al, Advances in Pharmacology, Volume 74 Copyright #2015 Elsevier Inc. ISSN 1054-3589, http://dx.doi.org/10.1016/bs.apha.2015.03.004,

### V

Valbenazine (activated by CYP3A4/5 to NBI-136110): https://go.drugbank.com/drugs/DB11915,
Vicagrel (activated by CYP450 enzymes): Future Med Chem. 2013 February; 5(2): 213-228. doi:10.4155/fmc.12.197, Shan J, Zhang B, Zhu Y, et al. Overcoming clopidogrel resistance: discovery of vicagrel as ahighly potent and orally bioavailable antiplatelet agent. J Med Chem 2012; 55:3342-3352, PubMed: 22428882.

The products obtained according to the invention contain at least one chemical substance, a mixture of substances, but at least one drug or active ingredient. The chemical substances are preferably an organic molecule which, in addition to carbon (C) and hydrogen (H), may contain heteroatoms, such as oxygen (O), nitrogen (N), sulfur (S) or phosphorus (P). The chemical substances may have linear and/or ring-shaped carbon chains including heteroatoms. Preferred are organic molecules with less than 1,000 g/mol, in particular less than 750 g/mol, less than 500 g/mol, less than 250 g/mol. Furthermore, it is preferred that at least one chemical substance contains at least one chiral carbon atom and represents a stereoisomer.

A prodrug according to the invention or the products obtained according to the invention can be sufficiently analyzed, for example, by combined analytical methods, such as GC/LC-MS, IR, NMR and MS, and, if necessary, subjected to structural elucidation.

A “peroxygenase” in the sense of the present invention is an enzyme capable of catalyzing one or more biochemical reactions. A peroxygenase according to the invention is capable of obtaining from a prodrug (reactants)—a substrate—a first (enzyme) product or products, in particular a drug.

The peroxygenases selected according to the invention are preferably of fungal origin, in particular nonspecific peroxygenases (“UPOs”, EC 1.11.2.1 (see enzyme.expasy.org)) Particularly preferred are peroxygenases from the fungal kingdom, especially from the dykarya subkingdom, in particular from the Basidiomycota and Ascomycota divisions.

Preferred nonspecific peroxygenases are derived from fungi of the order Agaricales, preferably from the families Psathyrellaceae, Marasmiaceae and Strophariaceae, in particular the genus Agrocybe, such species as Agrocybe aegerita and Agrocybe parasitica, the genus Psathyrella, such species as Psathyrella aberdarensis, and the genus Marasmius, such species as Marasmius rotula and Marasmius wettsteinii.

These peroxygenases according to the invention catalyze reactions like the P450 monooxygenases, whereas only an oxidizing agent, such as hydroperoxide (R—OOH) in particular hydrogen peroxide (H₂O₂) is required as cosubstrate for oxyfunctionalization of a substrate (A):

A+R—OOH UPO>AO+ROH

Furthermore, organic hydroperoxides (R—OOH, e.g. tert-butyl hydroperoxide), peroxycarboxylic acids (R—CO—OOH, e.g. meta-chloroperbenzoic acid) or hydrogen peroxide adducts (e.g. carbamide peroxide) can be used.

Reaction control using UPOs preferred according to the invention has advantages over P450 enzymes in vitro, in particular high water solubility due to the strong glycosylation of the proteins. As a result, such UPOs exhibit high stability compared to intracellular mono- or dioxygenases (free dissolved or membrane bound). UPOs, in contrast to intracellular systems, can be used advantageously under non-sterile conditions.

In a further embodiment, a peroxygenase may preferably be obtained from non-human organisms, such as fungi, yeasts, algae or bacteria.

The peroxygenases can be isolated and purified from organisms by known methods. Furthermore, such peroxygenases can be produced recombinantly in a host.

The invention is explained below using clopidogrel as an example.

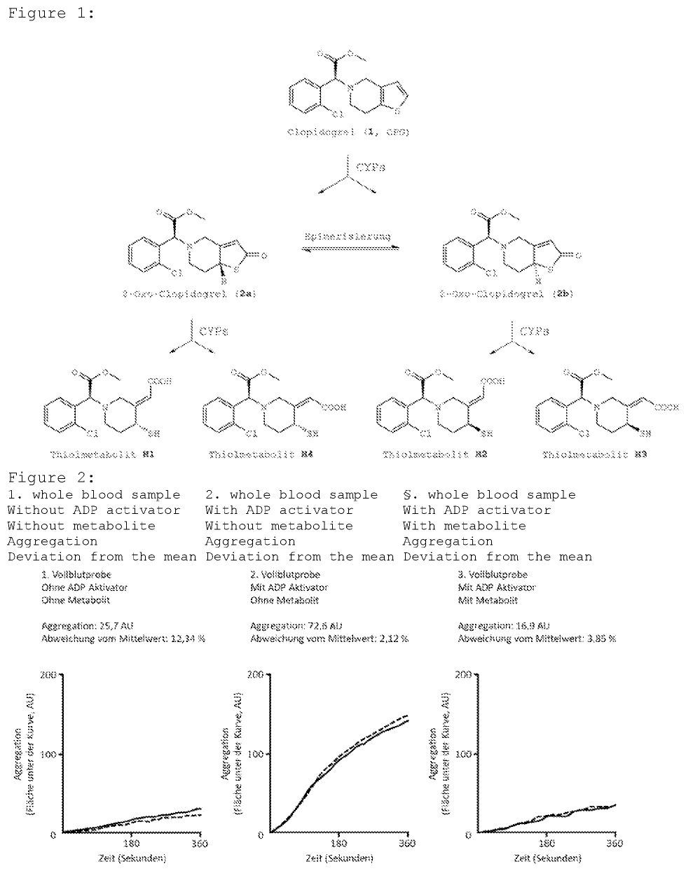

The two-step oxyfunctionalization of thienopyridine/clopidogrel according to a process of the invention selectively yields the desired thiol metabolites (H1-H4, FIG. 1), such as H3 and H4 with a high yield of 20%.

In a preferred embodiment, the peroxygenases, in particular UPOs are used at a low concentration of 0.1 U^{mL-1} to 10 U^{mL-1}. 1 to 3 U^{mL-1} for the oxyfunctionalization of the prodrug, in particular thienopyridines (1 unit converts 1 μmol of veratryl alcohol per minute).

In a preferred embodiment, the concentration of a prodrug according to the invention, in particular thienopyrdine is 0.1 to 10 mM, preferably 0.2 to 5 mM, more preferably between 0.5 to 2 mM, in particular 0.8 to 1.5 mM.

In a preferred embodiment of the invention, the prodrug is at least one thienopyridine. Such known thienopyridines are ticlopidine (IUPAC: (5-(2-chlorobenzyl)-4,5,6,7-tetrahydrothieno[3,2-c]pyridine), clopidogrel (IUPAC: (S)-methyl-(2-chlorophenyl)-2-(6,7-dihydro-4H-thieno[3,2-c]pyridin-5-yl)acetate, vicagrel (IUPAC: (S)-methyl-(2-chlorophenyl)-2-(2-acetyloxy-6,7-dihydro-4H-thieno[3,2-c]pyridin-5-yl)acetate), prasugrel (IUPAC: (S)-[5-[2-cyclopropyl-1-(2-fluorophenyl)-2-oxoethyl]-6,7-dihydro-4H-thieno[3,2-c]pyridin-2-yl]acetate) and tipidogrel (IUPAC: (5-(2-cyanobenzyl)-4,5,6,7-tetrahydrothieno[3,2-c]pyridine).

These thienopyridines are agents of the platelet aggregation inhibitor group and are used for the treatment and prophylaxis of both atheorothrombotic events, such as myocardial infarction, ischemic stroke, or peripheral artery disease, and other pharmacological effects, including. including stimulation of nitric oxide production, inhibition of erythrocyte aggregation, and reduction of circulating fibrinogen levels (Jolanta Siller-Matula, Karsten Schror, Johann Wojta, Kurt Huber, Thienopyridines in cardiovascular disease: Focus on Clopidogrel resistance Thromb. Haemost., 2007, 93(3), 385-393).

For the purposes of the present invention, the thienopyridines are “prodrugs with bioactivation by cytochrome P450 enzymes” or a prodrug, with their anticoagulant activity occurring only after hepatic bioactivation by cytochrome P450 enzymes. The active metabolites of thienopyridines mediate potent antitrombotic activity through highly specific, irreversible inhibition of the platelet P2Y12 receptor.

In the case of ticlopidine and clopidogrel, hydroxylation of the C2 carbon by P450 enzymes occurs first, followed by rearrangement to the 2-oxo metabolite. This intermediate is generated in vivo by rapid de-esterification in the case of prasugrel and vicagrel.

In all cases, the 2-oxo metabolite is converted to the active thiol metabolite by ring opening (Formula III below), with oxidative conversion by cytochrome P450 enzymes in each case.

The free thiol group of the active metabolites form disulfide bridges with the extracellular cysteine residues (Cys17 and Cys270) on the P2Y12 receptor, thereby inactivating the receptor. Thus, binding of the intrinsic substrate ADP is blocked and ADP-induced platelet aggravation is inhibited.

In particular, the incidence of neutropenia and the gastrointestinal side effects associated with ticlopidine have led to the development of clopidogrel, which has a superior safety and tolerability profile compared with ticlopidine (Siller-Matula et al., supra).

Clopidogrel (CPG, 1) is a prodrug that requires bioactivation via a two-step cytochrome P450-dependent oxidation to a pharmacologically active human metabolite and is shown in FIG. 1 and FIG. 1.

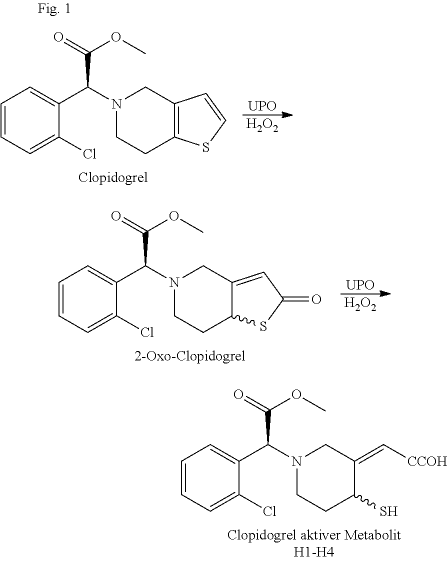

CPG (1, FIG. 1) is first oxidized to 2-oxo-clopidogrel (2-oxo-CPG, 2, FIG. 1) (see formula II below), which is present in two configurations by epimerization. Further oxidation of the thiolactone leads to the formation of a sulfenic acid intermediate, which is very unstable and is reduced directly by ascorbate to the active thiol or indirectly by glutathione (GSH) via a disulfide conjugate and subsequent reduction by another GSH molecule. Based on stereochemistry, the thiol metabolite can exist in four isomeric structures (H1-H4) (Drug Metabol. Dispos., 2012, 43(10), 1632-1641).

The antithrombotic activity is exclusively due to H4 (Tuffal, Gilles et al, An improved method for specific and quantitative determination of the clopidogrel active metabolite isomers in human plasma, Thromb. Haemost., 2011, 105(4), 696-705).

Prior art chemical syntheses for the preparation of 2-oxo-CPG (2, FIG. 1) have been described, but direct oxidation methods using hydrogen peroxide or mCPBA are complicated by the presence of the basic piperidine amine and do not yield the desired thiolactone (2a, 2b, FIG. 1).

In particular, the problem of direct utilization, namely to establish highly active P450 systems for regioselective oxidation and subsequent ring opening of thienopyridines without hydrolytic side reactions, remains unsolved.

Previously described processes lead to low yields, strong by-product formation and high isolation efforts due to complex reaction mixtures.

Therefore, the invention further relates to such prodrugs with bioactivation by cytochrome P450 enzymes according to formula I:

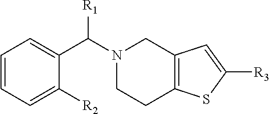

with independently

R1: Hydrogen, COOCH₃, COC3H5;

R2: Hydrogen, halogen, Cl, F, CN;

R3: Hydrogen, OH, OCOCH₃

in a process according to the invention.

According to formula I

Ticlopidine: R1=H, R₂=Cl, R₃=H;

Clopidogrel: R₁=COOCH₃, R₂=Cl, R₃=H;

Vicagrel: R₁=COOCH, R₃₂=Cl, R₃=OCOCH₃;

Prasugrel: R₁=COC H₃₅, R₂=F, R₃=OCOCH₃;

Tipidogrel: R₁=H, R₂=CN, R₃=OCOCH₃;

In addition, the invention relates to such products, which can be obtained from formula I according to the invention, of formula II

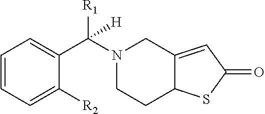

with independently

R1: Hydrogen, COOCH₃, COC₃H₅;

R2: Hydrogen, halogen, Cl, F, CN;

or of the formula III, so-called 4-mercapto-3-piperidinylideneacetic acid derivatives

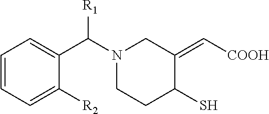

with independently

R1: Hydrogen, COOCH₃, COC₃H₅;

R2: Hydrogen, halogen, Cl, F, CN.

The process according to the invention can advantageously be carried out in a one-pot device (reactor) for a prodrug according to the invention.

The one-pot device can be equipped with stirrers and other common utilities.

The process according to the invention can be carried out particularly preferably in aqueous, in particular buffered solutions with buffer concentrations of 1 to 100 mM, preferably 10 to 20 mM. The reaction process is carried out at pH values of 3 to 10, preferably 5 to 8, in particular at pH 7. The reaction can be carried out without solvent or in the presence of a solvent miscible with water, for example alcohols, acetone, and acetonitrile or in a two-phase system with, for example, dichloromethane, but preferably with acetone or acetonitrile in the ranges 1-90 vol %, particularly preferably 2-50 vol %, especially 5-30 vol %.

Compared to the natural P450 system, electron transport proteins and regulatory proteins (flavin reductases, ferredoxins) can be omitted in the process according to the invention.

In a further preferred embodiment of the invention, the process is carried out in the presence of radical scavengers. For example, ascorbic acid or glutathione can be used as radical scavengers. In the case of clopidogrel, for example, when ascorbic acid is used, H3 and H4 can be formed selectively and in equal amounts. When glutathione is used, H1-H4 are formed in equal parts. The concentration can be 0.01 mM to about 100 mM, especially 5 to 10 mM.

In another preferred embodiment of the invention, the process is carried out at normal pressure and temperatures of 4-0°, preferably at 15-35° C. and most preferably at 20-25° C.

The isolation of the obtained products or intermediates can be carried out by means of usual purification, e.g. from the supernatant, for example by extraction, filtration, distillation, rectification, chromatography, treatment with ion exchangers, adsorbents or crystallization. Preferably, the products are isolated by liquid-liquid extraction and purified chromatographically. The obtained products or chemical compounds can be identified e.g. by LC/MS.

The following examples and figures serve to explain the invention in more detail, without, however, limiting the invention to these examples and figures.

### EXAMPLES AND FIGURES

### Example 1

For the enzymatic synthesis of the active clopidogrel metabolites (IUPAC: 2-(1-((S)-1-(2-chlorophenyl)-2-methoxy-2-oxoethyl)-4-mercaptopiperidin-3-ylidene)acetic acid), 100 mg clopidogrel hydrogen sulfate (0.24 mmol, final 1.2 mM) was dissolved in 40 mL acetone, 138 mL water, and 20 mL 200 mM potassium phosphate buffer (pH 7, final 20 mM). To the suspension were added 350 mg ascorbic acid and 600 U MroUPO (units=μmol^{min-1} based on the oxidation of veratryl alcohol to veratrylaldehyde) (final concentration of 3 U^{mL-1}). The mixture was stirred at 25° C. and 200 rpm. Using a syringe pump, a 400 mM hydrogen peroxide solution was continuously added at a rate of 1 mL/h (2 mM/h). The progress of the reaction was continuously monitored by DC and HPLC. After a reaction time of 150 min, the reaction was stopped by extracting the products with three times 30 mL of dichloromethane. The organic phases were combined, dried with sodium sulfate and concentrated to dryness. The yellowish crude product was taken up in methanol and purified by preparative HPLC using a semi-preparative column (LiChrospher® 100 column, LiCART® 250×10 mm, RP-18e, 10 μm, Merck KGaA, Darmstadt, Germany). The acetonitrile from the fractions was evaporated and the products were subsequently dried by lyophilization.

Clopidogrel active metabolites H3 and H4

Yield: 15 mg (18%), purity (>95%, HPLC-ELSD).

HRMS (ESI+): m/z calculated for C16H19ClNO4S [M+H]+: 355.0718, found: 356.0718 [M+H]+(100%)

### Example 2

Example of embodiment of the effect of a metabolite produced via peroxygenase on human platelets (blood platelets).

The metabolite is present as a powder (approx. 4 mg) in a dark container and is stored in a freezer at at least −80° C. until the solution is prepared. On the day of use, the drug is dissolved in 500 μl of a suitable sterile solvent (here: phosphate-buffered saline, PBS). Powder and solvent are mixed rigorously. The complete dissolution of the powder is to be visually checked. The mixture is stored on ice or at approx. 0° C. until use.

To test the functionality of the agent, the aggregation of human thrombocytes (platelets) in whole blood is determined by impedance aggregometry (Paniccia, R.; Antonucci, E.; Maggini, N.; Romano, E.; Gori, A. M.; Marcucci, R.; Prisco, D.; Abbate, R. Assessment of Platelet Function on Whole Blood by Multiple Electrode Aggregometry in High-Risk Patients with Coronary Artery Disease Receiving Antiplatelet Therapy. American journal of clinical pathology 2009, 131, 834-42, doi:10.1309/AJCPTE3K1SGAPOIZ). For the test, approximately 5 ml of fresh whole blood is collected from a visible vein at the elbow (cubital vein) of a subject by an appropriately trained person using appropriate sterile blood collection equipment. The blood is collected into a collection vessel designed for this purpose (e.g. Sarstedt Monovette, Sarstedt, Numbrecht, Germany). This contains e.g. tri-sodium citrate in a standard concentration of 0.106 mol-L-1.

Adenosine diphosphate (ADP, 10 μM final concentration) induced platelet aggregation testing was performed according to the Multiplate® Analyzer manufacturer's instructions (Roche Diagnostics, Mannheim, Germany).

At least three different samples are tested:

### Spontaneous Whole Blood Sample

Whole blood without activation by ADP. The volume of activator and drug—20 μL each—is replaced by a phosphate-buffered saline solution.

ADP Activated Whole Blood Sample without Active Substance

The volume of active substance is replaced by a phosphate-buffered saline solution.

ADP activated whole blood sample with active substance Whole blood activated by ADP. The active substance is added with the above volume.

FIG. 2 shows representatively the reduction of ADP-induced platelet aggregation during a two-minute pre-incubation of the metabolite in the blood of an apparently healthy subject. The theoretical initial concentration of the metabolite in this experiment was 9.40 mg per 1 mL of PBS solvent (4.70 mg of metabolite with approximately 73% purity in 500 μL of PBS solution). The final concentration of the metabolite in the tested blood was 0.31 mg per 1 mL (20 μL of metabolite solution in 600 μL blood sample).

### Example 3

Implementation of Tamoxifen with UPOs

Reaction of 1 mM tamoxifen (citrate salt) was performed with seven different UPOs (20 UABTS/mL for rAaeUPO, MroUPO, CglUPO; 2-3 U/mL for rPabUPO and M576UPO) in the phosphate-buffered system (20 mM, pH 7) in the presence of ascorbic acid (5 mM) and hydrogen peroxide (2 mM, added 4 times every 15 min) at 30° C. and 800 rpm for 1 h. The reaction was performed in the presence of a phosphate buffer. Samples were processed for MS analysis and analyzed using Orbitrap.

Here, the CglUPO and M576UPO showed the best conversion rates with 40-45% under non-optimized conditions. Both UPOs formed N-desmethyltamoxifen as well as various hydroxylated products.

A total of four different hydroxytamoxifen derivatives were found (retention times: 3.77, 4.22, 4.35, 4.81). Based on the retention times, N-desmethyl-OH-tamoxifen (retention time 3.67) should be the demethylated product of hydroxytamoxifen (retention time 3.77), which occurs very dominantly with CglUPO. Similarly, N-desmethyl-OH-tamoxifen (4.18) as the demethylation product of hydroxytamoxifen (4.22) with M576UPO.

1. A pharmaceutical composition for use in the treatment of patients whose P450 enzyme set or oxidoreductases are impaired by mutations or polymorphism, prepared by a process characterized in that at least one peroxygenase is added to a liver-specific prodrug having bioactivation by cytochrome P450 enzymes and the product obtained is isolated, wherein
i) the peroxygenase is selected from the group consisting of fungi, yeasts, algae, and bacteria,
ii) the prodrug is selected from the group Amiodarone, Aripiprazole lauroxil, Bambuterol, Brincidofovir, Carisoprodol, Clomifene, Codeine, Decarbazine, N-Desmethyltamoxifen, Desogestrel, N,N-Didesmethyltamoxifen, Ellipticine, Enalapril, Fludrocortisone, Flutamide, Irinotecan, Lansoprazole, Leflunomide, Levomethorphan, Loratadine, Losartan, Lynestrenol, Nabumetone, Pantoprazole, Parecoxib, Primidone, Proguanil, Sibutramine, Tafenoquine, Tegafur, Temozolomide, Tramadol, Treosulfan, and Valbenazine, or
iii) the prodrug is at least one thienopyridine, in particular of formula I 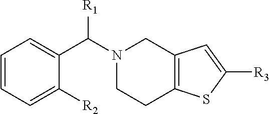
where independently of one another
R₁: hydrogen, COOCH₃, COC₃H₅;
R₂: hydrogen, halogen, Cl, F, CN; and
R₃: hydrogen, OH, OCOCH₃.

2. The pharmaceutical composition for use according to claim 1, wherein the thienopyridine is selected from the group consisting of ticlopidine (IUPAC: (5-(2-chlorobenzyl)-4,5,6,7-tetrahydrothieno[3,2-c]pyridine), clopidogrel (IUPAC: (S)-methyl-(2-chlorophenyl)-2-(6,7-dihydro-4H-thieno[3,2-c]pyridin-5-yl)acetate, vicagrel (IUPAC: (S)-methyl-(2-chlorophenyl)-2-(2-acetyloxy-6,7-dihydro-4H-thieno[3,2-c]pyridin-5-yl)acetate), prasugrel (IUPAC: (S)-[5-[2-cyclopropyl-1-(2-fluorophenyl)-2-oxoethyl]-6,7-dihydro-4H-thieno[3,2-c]pyridin-2-yl]acetate), and tipidogrel (IUPAC: (5-(2-cyanobenzyl)-4,5,6,7-tetrahydrothieno[3,2-c]pyridine).

3. The pharmaceutical composition for use according to claim 1, wherein the prodrug with bioactivation by cytochrome P450 enzymes is present as an isolated substance or pure substance.

4. The pharmaceutical composition for use according to claim 1, wherein the prodrug having bioactivation by cytochrome P450 enzymes is not present in a liver cell.

5. The pharmaceutical composition for use according to claim 1, wherein a regioselective, enzymatic oxyfunctionalization of the prodrug is carried out by means of an oxidizing agent.

6. The pharmaceutical composition for use according to claim 1, wherein the oxidizing agent is selected from the group consisting of hydroperoxide, hydrogen peroxide, peroxycarboxylic acids, and hydrogen peroxide adducts.

7. The pharmaceutical composition for use according to claim 1, wherein the peroxygenase is selected from the group consisting of non-specific peroxygenases and the enzyme class EC 1.11.2.1.

8. A medicament for use according to claim 1, wherein the peroxygenase is selected from fungi, in particular of the order Agaricales, families Psathyrellaceae, Marasmiaceae and Strophariaceae, genus Agrocybe, in particular Agrocybe aegerita, Agrocybe parasitica, genus Psathyrella, in particular Psathyrella aberdarensis, genus Marasmius, in particular Marasmius rotula, Marasmius wettsteinii.

9. The pharmaceutical composition for use according to claim 1, wherein the process is carried out in buffered solutions at pH values of 3 to 10.

10. The medicament for use according to claim 8, wherein the process is carried out at temperatures of 4-40° C., in particular of 15-25° C.

11. The pharmaceutical composition for use according to claim 1, wherein the isolated product is according to formula II 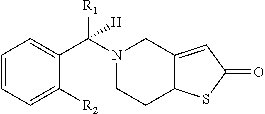
where independently
R₁: hydrogen, COOCH₃, COC₃H₅; and
R₂: hydrogen, halogen, Cl, F, CN;
and/or the formula III 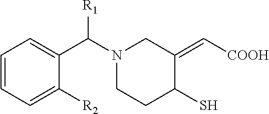
where independently
R₁: hydrogen, COOCH₃, COCH₃₅; and
R₂: hydrogen, halogen, Cl, F, CN.

12. The pharmaceutical composition for use according to claim 1, wherein at least one enzyme is selected from the group consisting of CYP1A1, CYP1A2, CYP2A6, CYP2B6, CYP2C8, CYP2C9, CYP2C19, CYP2D6, CYP2E1, CYP3A4, CYP3A5, CYP3A7, CYP4A11, CYP1B1, CYP2A13, CYP2F1, CYP2J2, CYP2R1, CYP2S1, CYP2U1, CYP2W1, and CYP3A43.

13. A medicament prepared by the process according to claim 1.
